(19) United States
(12) Patent Application Publication
(10) Pub. No.: US 2008/0215140 A1
(43) Pub. Date: Sep. 4, 2008

(54) STENTS COATED WITH BIOMOLECULES AND PROCESS FOR THEIR PRODUCTION

(75) Inventors: Alexander Borck, Aurachtal (DE); Nina Adden, Braunschweig (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/018,295

(22) Filed: Jan. 23, 2008

(30) Foreign Application Priority Data
Jan. 25, 2007 (DE) 10 2007 003 708.4

(51) Int. Cl. A61F 2/82; A61L 33/04; A61K 47/24

(52) U.S. Cl. 623/1.46; 427/2.25; 514/772

(57) ABSTRACT

A stent comprising a basic stent body, at least one anchor group on the surface of the basic stent body, and at least one biomolecule which is bound to the at least one anchor group, the anchor group being the same or different and selected from the group of compounds with the general formula (I)

(R²O)₂(O)P-L-(CH₂)ₓ-M-R¹ (I)

in which
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂—CH₂—O)_{y},
x represents an integer from 1 to 25,
y represents an integer from 1 to 25
   and the biomolecule or biomolecules are selected as the same or different from the group consisting of compounds which promote the attachment of the endothelial progenitor cells to the stent surface.

### PRIORITY CLAIM

This patent application claims priority to German Patent Application No. 10 2007 003 708.4, filed Jan. 25, 2007, the disclosure of which is incorporated herein by reference in its entirety.

### FIELD

The present disclosure relates to a stent comprising a) a basic stent body, b) one or several anchor groups on the surface of the basic stent body, and c) one or several biomolecules which are bound to the anchor group or anchor groups. The present disclosure also relates to a process for the production of a stent according to the disclosure; and the use of compounds with the general formula (I) (R²O)₂(O)P-L-(CH₂)ₓ-M-R¹ (the substituents being described in the following) as anchor groups for biomolecules of basic stent bodies and as anchor groups for the production of one or several stents according to the disclosure.

### BACKGROUND

Stents, in general, are endovascular prostheses or implants which are used for treating stenoses, for example. In addition, stents are known for the treatment of aneurism. Basically, stents have a carrier structure suitable for supporting the wall of a vessel in an appropriate manner in order to enlarge the vessel and/or to bridge an aneurism. Stents are introduced into the vessel for this purpose in a compressed state and then are expanded at the site to be treated and pressed into the vessel wall. This expansion can take place by means of a balloon catheter. As an alternative, self-expanding stents are also known. Self-expanding stents contain, for example, a super-elastic metal such as nitinol.

At present, stents are divided into two basic types: permanent stents and biodegradable stents. Permanent stents are designed in such a way that they can remain in the vessel for an indefinite period. Biodegradable stents, on the other hand, are degraded in a vessel over a predetermined period. Preferably, biodegradable stents are degraded only once the traumatized tissue of the vessel has healed and, consequently, the stent no longer needs to remain in the vessel lumen.

However, it has been found that by introducing stents into vessel systems, secondary effects such as restenosis and thromboses may occur.

### SUMMARY

The present disclosure describes several exemplary embodiments of the present invention.

One aspect of the present disclosure provides a stent comprising a) a basic stent body; b) at least one anchor group on the surface of the basic stent body; and c) at least one biomolecule which is bound to the anchor group or groups wherein the at least one anchor group is selected as the same or different from the group of compounds consisting of the general formula (I)

(R²O)₂(O)P-L-(CH₂)ₓ-M-R¹ (I)

wherein R¹ represents —COOH, —OH, —SH, —NH₂, benzophenone or benzophenone derivatives, R² represents hydrogen, —CH₂CH₃ or —CH₃, L represents a single bond or —O—, M represents a single bond or —(CH₂—CH₂—O)_{y}, x represents an integer selected from the group consisting of 1 to 25 and y represents an integer selected from the group consisting of 1 to 25.

Another aspect of the present disclosure provides a method for producing a stent coated with biomolecules, the method comprising a) providing at least one basic stent body; b) purifying the at least one basic stent body; and c) selecting at least one identical or different anchor group selected from the group of compounds consisting of the general formula (I)

(R²O)₂(O)P-L-(CH₂)ₓ-M-R¹ (I)

wherein R¹ represents —COOH, —OH, —SH, —NH₂, benzophenone or benzophenone derivatives, R² represents hydrogen, —CH₂CH₃ or —CH₃, L represents a single bond or —O—, M represents a single bond or —(CH₂—CH₂—O)_{y}, x represents an integer selected from the group consisting of 1 to 25, and y represents an integer selected from the group consisting of 1 to 25, d) functionalizing the purified basic stent body from b) with at least one identical or different anchor groups selected from the group of compounds with the general formula (I) from c); e) providing at least one identical or different biomolecules from the group consisting of compounds which promote the attachment of the endothelial progenitor cells to the stent surface; and f) binding at least one identical or different biomolecules from e) to the functionalized basic stent body or bodies from d).

A further aspect of the present disclosure provides an anchor group for biomolecules on basic stent bodies, comprising a compound with the general formula (I)

(R²O)₂(O)P-L-(CH₂)ₓ-M-R¹ (I)

in which
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂, —CH₂—O)_{y},
x represents an integer from 1 to 25, and
y represents an integer from 1 to 25.

An additional aspect of the present disclosure provides a method for producing at least one stent, comprising providing at least one stent, incorporating at least one anchor group comprising a compound with the general formula (I)

(R²O)₂(O)P-L-(CH₂)ₓ-M-R¹ (I)

in which
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂—CH₂—O)_{y},
x represents an integer from 1 to 25, and
y represents an integer from 1 to 25.

One feature of the present disclosure provides a stent which involves a reduced risk of restenosis on introduction into the vessel system in comparison with stents of the state of the art.

One feature of the present disclosure is achieved by a stent comprising: a basic stent body; at least one anchor group on the surface of the basic stent body; and at least one biomolecule which is bound to the anchor group or groups wherein the at least one anchor group is selected as the same or different from the group of compounds consisting of the general formula (I)

(R²O)₂(O)P-L-(CH₂)ₓ-M-R¹ (I)

wherein
R¹ represents —COOH, —OH, —SH, —NH₂, benzophenone or benzophenone derivatives, preferably —COOH or —NH₂,
R² represents hydrogen, —CH₂CH₃ or —CH₃, preferably hydrogen,
L represents a single bond or —O—, preferably a single bond,
M represents a single bond or —(CH₂—CH₂—O)_{y}, preferably a single bond,
x represents an integer selected from the group consisting of 1 to 25, preferably 3, 4, 5, 6, 7, 8, 9 or 10 and
y represents an integer selected from the group consisting of 1 to 25, preferably 3, 4, 5, 6, 7, 8, 9, 10, 11 or 12
   and their salts and hydrates; and the biomolecule or biomolecules are selected as the same or different from the group consisting of compounds which support the attachment of the endothelial progenitor cells to the stent surface.

In this respect, the preferred embodiments of the stent may be present all together, partially, in any desired combination, and individually.

A further feature of the present disclosure provides a method for producing a stent, the method comprising:
a) providing at least one basic stent body;
b) purifying the at least one basic stent body;
c) selecting at least one identical or different anchor group from the group of compounds consisting of the general formula (I)

(R²O)₂(O)P-L-(CH₂)ₓ-M-R¹ (I)

wherein
R¹ represents —COOH, —OH, —SH, —NH₂, benzophenone or benzophenone derivatives, preferably —COOH or —NH₂,
R² represents hydrogen, —CH₂CH₃ or —CH₃, preferably hydrogen,
L represents a single bond or —O—, preferably a single bond,
M represents a single bond or —(CH₂—CH₂—O)_{y}, preferably a single bond,
x represents an integer selected from the group consisting of 1 to 25, preferably 3, 4, 5, 6, 7, 8, 9 or 10 and
y represents an integer selected from the group consisting of 1 to 25, preferably 3, 4, 5, 6, 7, 8, 9, 10, 1 or 12
   and their salts and hydrates being provided;
d) functionalizing the purified basic stent body from b) with at least one identical or different anchor groups selected from the group of compounds consisting of the general formula (I) from c);
e) providing at least one identical or different biomolecule selected from the group consisting of compounds which promote the attachment of the endothelial progenitor cells to the stent surface; and
f) binding at least one identical or different biomolecule from e) to the functionalized basic stent body or bodies from d).

In this respect, the preferred embodiments of the anchor groups may be present all together, partially, in any desired combination, and individually.

A further embodiment of the present disclosure uses at least one compounds selected from the group consisting of the general formula (I)

(R²O)₂(O)P-L-(CH₂)ₓ-M-R¹ (I)

wherein
R¹ represents —COOH, —OH, —SH, —NH₂, benzophenone or benzophenone derivatives preferably —COOH or —NH₂,
R² represents hydrogen, —CH₂CH₃ or —CH₃, preferably hydrogen,
L represents a single bond or —O—, preferably a single bond,
M represents a single bond or —(CH₂—CH₂—O)_{y}, preferably a single bond,
x represents an integer selected from the group consisting of 1 to 25, preferably 3, 4, 5, 6, 7, 8, 9 or 10 and
y represents an integer selected from the group consisting of 1 to 25, preferably 3, 4, 5, 6, 7, 8, 9, 10, 11 or 12
   and their salts and hydrates as anchor groups for biomolecules on basic stent bodies.

In this respect, exemplary embodiments of the anchor group may be present all together, partially, in any desired combination, and individually.

In addition, a further exemplary embodiment of the present disclosure uses compounds with the general formula (I)

(R²O)₂(O)P-L-(CH₂)ₓ-M-R¹ (I)

wherein
R¹ represents —COOH, —OH, —SH, —H₂, benzophenone or benzophenone derivatives, preferably —COOH or —NH₂,
R² represents hydrogen, —CH₂CH₃ or —CH₃, preferably hydrogen,
L represents a single bond or —O—, preferably a single bond,
M represents a single bond or —(CH₂—CH₂—O)_{y}, preferably a single bond,
X represents an integer selected from the group consisting of 1 to 25, preferably 3, 4, 5, 6, 7, 8, 9 or 10 and
Y represents an integer selected from the group consisting of 1 to 25, preferably 3, 4, 5, 6, 7, 8, 9, 10, 11 or 12
   and their salts and hydrates as anchor groups for the production of one or several stents according to the disclosure.

In this respect, the exemplary embodiments of the anchor group may be present all together, partially, in any desired combination, and individually.

### DETAILED DESCRIPTION

The present invention is based on the finding that by way of the coating, according to the disclosure, of the stents with biomolecules, endothelial progenitor cells (EPCs) are bound to the stent surface. This leads to the stent surface being colonized more rapidly with endothelial cells (EC) in comparison with a stent of the prior art. As a result, the endothelial layer of the vessel is built up more rapidly in the area of stent application, thus, the risk of restenosis is reduced. The rapid and complete colonization with endothelium prevents late thromboses such as can be observed, in particular, in the case of stents releasing active principle. The feature common to stents releasing active principle which are known in the prior art is that the stents use polymers or active principles which prevent endothelialization and thus lead to the late thromboses described above.

Surprisingly enough, it has been recognized that, by using compounds with the general formula (I)

(R²O)₂(O)P-L-(CH₂)ₓ-M-R¹ (I)

wherein R¹ represents —COOH, —OH, —SH, —NH₂, benzophenone or benzophenone derivatives, R² represents hydrogen, —CH₂CH₃ or —CH₃, L represents a single bond or —O—, M represents a single bond or —(CH₂—CH₂—O)_{y}, x represents an integer selected from the group consisting of 1 to 25 and y represents an integer selected from the group consisting of 1 to 25 as anchor groups on the basis stent bodies, the risk of inflammation and/or the risk of thrombosis is reduced in comparison with polymers. It is believed that inflammations and/or thromboses are caused by the degradation of the polymers, e.g., polyurethanes or some polyesters. In contrast, the anchor compounds according to the present disclosure selected from the group of compounds with the general formula (I) are not degraded in an inflammation and/or thrombosis promoting manner under physiological conditions.

As a result of the stent according to the present disclosure, it is moreover possible to make use of natural mechanisms of the body in order to repair the damaged endothelial layers of the vessel, preferably in the area of stent application. These natural mechanisms of the body include, among others, the following body-inherent mechanisms: generation of more rapid and better targeted growth of endothelium. As a result of the cell layer being closed early, fewer signal molecules, such as inflammation mediators, are released.

A basic stent body according to the present disclosure should be understood to mean a permanent or degradable metal stent or a polymer stent.

### Permanent Metal Stent

The basic body of the stent preferably consists of a metal material of one or several metals selected from the group consisting of iron, magnesium, nickel, tungsten, titanium, zirconium, niobium, tantalum, zinc or silicon and, if necessary, a second component of one or several metals from the group consisting of lithium, sodium, potassium, calcium, manganese, iron or tungsten, preferably of a zinc-calcium alloy. In a further practical example, the basic body consists of a memory effect material of one or several materials from the group consisting of nickel titanium alloys and copper zinc aluminium alloys, but preferably of nitinol. In a further practical example, the basic body of the stent consists of stainless steel, preferably of a Cr—Ni—Fe steel, in this case, preferably the alloy 316L, or a Co—Cr steel. Moreover, the basic body of the stent may consist at least partially of plastic and/or a ceramic material.

### Degradable Metal Stent

Preferably, the biocorrodible metallic material is a biocorrodible alloy selected from the group consisting of magnesium, iron and tungsten; the biocorrodible metallic material is, in particular, a magnesium alloy.

The alloys of the elements magnesium, iron or tungsten should be selected with respect to their composition in such a way that the alloys are biocorrodible. For purposes of the present disclosure, alloys are referred to as biocorrodible when degradation occurs in a physiological environment which, in the end, leads to the entire implant or the part of the implant formed of the material losing its mechanical integrity.

For purposes of the present disclosure, the term alloy should be understood to mean a metallic structure whose main component is magnesium, iron or tungsten. The main component is the alloy component whose proportion by weight of the alloy is greatest. A proportion of the main component preferably amounts to more than 50% by weight, in particular, more than 70% by weight.

If the material is a magnesium alloy, the material preferably contains yttrium and other rare earth metals since the remarkable features of such an alloy are its physical-chemical properties and high biocompatibility, in particular, also of its degradation products.

Particularly preferably, a magnesium alloy with the composition of: rare earth metals 5.2-9.9% by weight, including yttrium of 0.0-5.5% by weight, the remainder being <1% by weight is used, magnesium representing the remaining part of the alloy to make 100% by weight. This magnesium alloy has already confirmed its particular suitability in experiments and in initial clinical trials, i.e., the magnesium alloy exhibits a high biocompatibility, advantageous processing properties, good mechanical characteristic values and a corrosion behaviour adequate for the purposes of use. For purposes of the present disclosure, the collective term “rare earth metals” should be understood to mean scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

### Permanent Polymer Stent

Basic stent bodies of permanent polymer stents preferably consist of polypropylene, polyethylene, polyvinyl chloride, polymethyl methyl ethyl acrylate, polymethyl ethyl acrylate, polytetrafluoroethylene, polyvinyl alcohol, polyurethane, polybutylene terephthalate, silicones, polyphosphates and their copolymers and blends or polyhydroxybutyric acid (atactic, isotatic, syndiotactic and their blends) and the like.

### Degradable Polymer Stent

Basic stent bodies of degradable polymer stents preferably consist of polydioxanone, polyglycolide, polycaprolactone, polylactide [poly-L lactide, poly-D,L lactide and copolymers and blends such as poly(L-lactide coglycolide), poly(D,L-lactide coglycolide), poly(L-lactide co-D,L-lactide), poly(I-lactide cotrimethylene carbonate)], triblock copolymers, polysaccharides [chitosan, levan, hyaluronic acid, heparin, dextran, cellulose etc.], polyhydroxyvalerate, ethylvinyl acetate, polyethylene oxide, polyphosphoryl choline, fibrin, albumin and the like.

Biodegradable and permanent metal stents are preferred to poly-stents.

According to the present disclosure, one or several anchor groups for a stent are selected the same or different from the group of compounds with the formula (I) wherein
R¹ represents —COOH, —OH, —SH, —NH₂, benzophenone or benzophenone derivatives, preferably —COOH or —NH₂,
R² represents hydrogen, —CH₂CH₃ or —CH₃, preferably hydrogen,
L represents a single bond or —O—, preferably a single bond,
M represents a single bond or —(CH₂—CH₂—O)_{y}, preferably a single bond,
x represents an integer selected from the group consisting of 1 to 25, preferably 3, 4, 5, 6, 7, 8, 9 or 10 and
y represents an integer selected from the group consisting of 1 to 25, preferably 3, 4, 5, 6, 7, 8, 9, 10, 11 or 12
   and their salts and hydrates.

Accordingly, compounds with the general formula (I) include phosphonic acids with the general formula (II)

(HO)₂(O)P—O(CH₂)ₓ-M-R¹ (II)

and their esters with the general formula (III)

(R²O₂(O)P-—(CH₂)ₓ-M-R¹ (III)

and compounds of phosphoric acid with the general formula (IV)

(HO)₂(O)P—O—(CH₂)ₓ-M-R¹ (IV)

and/or their esters with the general formula (V)

(R²O)₂(O)P—O(CH₂)ₓ-M-R¹ (V)

in which the preferred radicals R¹, R², M and x are defined according to the general formula (I) and the preferred radicals may be present all together, partially, in any desired combination, and individually.

Compounds with the general formulae (I), (II), (III), (IV) and (V), in which M represents an oligoethylene glycol linker —((CH₂)CH₂O)_{y}— are particularly preferred since, as a result, a non-specific protein attachment to these anchor groups is avoided in contrast to anchor groups in which M represents a single bond.

For purposes of the present disclosure, biomolecules are understood to mean those compounds which promote the attachment of endothelial progenitor cells to the stent surface and thus contribute to the endothelialization of the stent which, moreover, reduces the increased growth caused by the damage to the vessels of smooth muscle cells in vessels, preferably in the area of the stent application.

Suitable proteins as biomolecules are, preferably, e.g., protein A, enzymes, growth factors, antibodies, such as, e.g., CD 133, but also peptide sequences, such as, e.g., cyclic RGD. The antibody CD 133 and protein A are preferred since these offer a good starting base for binding antibodies in aligned manner.

The provision of a basis stent body according to the present disclosure should be understood to mean the provision of a non-derivatized and/or functionalised stent. Such basic stent bodies consist of the above stent materials and possess a geometry which is generally known in the art.

For purposes of the present disclosure, the purification step of basic stent bodies is intended to mean a treatment of the basic stent body which activates the basic stent body such that, during the subsequent step, the anchor molecules are bound to the basic stent body. For purposes of the present disclosure, preferably, the purification step is intended to mean purification in an oxygen plasma or by rinsing with solvent, preferably from the series of solvents consisting of dichloromethane, acetone, methanol and Millipore water. If necessary, the purification step can be followed by a drying step.

The provision of anchor groups selected from among groups with the general formula (I) is intended to mean a provision of these compounds dissolved, suspended or emulsified in a solvent selected from the group consisting of methanol, ethanol, acetone, tetrahydrofuran, dimethyl formamide, chloroform, dimethyl sulphoxide, dichloromethane etc., preferably dry tetrahydrofuran.

For purposes of the present disclosure, functionalization of the purified basic stent bodies with one or several identical or different anchor groups is intended to mean the entire or partial bringing into contact of the purified basic stent body with one or several solutions of the same and/or different anchor groups of the compounds with the formula (I), the solvent or solvents being subsequently evaporated entirely or partially, the solvent or solvents being, in particular, evaporated within one hour in such a way that the meniscus of the solution migrates over the stent surface. For purposes of the present disclosure, bringing into contact is intended to mean preferably spraying of the purified basic stent body with one or several solutions of the same and/or different anchor groups or immersion of the purified basic stent body into one or several solutions of the same and/or different anchor groups.

Subsequently, the stent functionalised with one or several anchor groups can, if necessary, be thermally treated (annealed), preferably over a period of 1 to 124 hours, preferably within a temperature range of 60 to 220° C. Preferably, annealing is carried out for 18 to 74 hours at 100 to 140° C.

If necessary, the functionalized stent, whether thermally treated or not, is subsequently rinsed with solvent.

Subsequently, the functionalized stent thus pretreated can, if necessary, be placed for a period of 1 to 24 hours into a carbonyl diimidazol solution (CDI) in dry dioxan, preferably for 15 hours into a 0.3 M solution of carbonyl diimidazol in dry dioxan. If necessary, this is followed by rinsing of the functionalized stent with dry dioxan.

Subsequently, a drying step in a stream of nitrogen can follow, if necessary.

The provision of biomolecules according to the disclosure is intended to mean a solution, suspension and emulsion of the biomolecules in a buffer solution which is free from amino acid. Suitable buffers are phosphate buffers, PBS buffers (phosphate buffered saline), MES buffers (2-morpholinoethane sulphonic acid), borate buffers and the like. The PBS buffer is preferred.

The functionalized stent or stents is/are brought into contact, fully or partially, with the solution, suspension or emulsion of the biomolecule in the buffer and, if necessary, rinsed therewith. For purposes of the present disclosure, brining into contact is intended to mean preferably spraying of the functionalised basic stent body with one or several buffer solutions of identical and/or different biomolecules or immersion of the purified basic stent body into one or several buffer solutions of the same and/or different biomolecules.

Subsequently, a drying step may follow, if necessary, by dry blowing with nitrogen.

For the formation of the bond between the biomolecules and benzophenone anchor groups, a step of exposure to light, e.g., at 260 nm with 100 mW/cm², is subsequently carried out.

For the formation of the bond of the biomolecules to carbonyl anchor groups, an activation with NHS/EDC and subsequent coupling of the biomolecule take place.

The present invention will be described by the following practical examples, characteristic features, details and advantages of the invention, in particular, arising therefrom. However, the present invention is not restricted to the practical examples.

### EXAMPLES

### Example 1

A stent purified in oxygen plasma and/or by rinsing with the solvents of the series dichloromethane, acetone, methanol and Millipore water is treated further as follows:

A 1 mM solution of hydroxyundecyl phosphonic acid in dry tetrahydrofuran is produced. The stent is suspended into this solution and the solvent is evaporated within one hour, the meniscus of the solution migrating over the stent surface.

Subsequently, the stent is annealed at 120° C. for 18 hours and then rinsed with solvent.

The stent thus pretreated is placed for 15 hours into a 0.3 M solution of carbonyl diimidazol (CDI) in dry dioxan. Subsequently, the stent is rinsed twice for 10 minutes with dry dioxan and subsequently dried in a stream of nitrogen.

A solution of CD 133 (approximately 50 μg/ml) in PBS buffer (free from amino acid) is placed onto the stent thus treated and shaken overnight at 4° C. Subsequently, the stent is rinsed with buffer.

The detection of bound protein takes place by mean of fluorescence labelled antigen CD 133.

### Example 2

A stent purified according to Example 1 is treated further as follows:

A 3 mM solution of 3-(4-oxybenzophenone) propyl phosphonic acid in dry tretrahydrofuran is produced.

The purified stent is rinsed three times with this solution. Subsequently, the stent is annealed for 12 hours at 120° C. and subsequently rinsed with solvent.

These stents are placed into a solution of CD 133 (approximately 500 μg/ml) in buffer and shaken overnight at 4° C.

Next day, the stents are removed from the solvent, dried and exposed to light at 260 nm with 100 mW/cm².

Non-bound protein is removed by washing.

The detection of bound proteins takes place by fluorescence-labelled antigen to CD 133.

### Example 3

A stent purified according to Example 1 is treated further as follows:

A 1 mM solution of carboxydodecyl phosphonic acid in dry tetrahydrofuran is produced.

The stent is suspended in this solution and the solvent is evaporated within one hour, the meniscus of the solution migrating over the stent surface. Subsequently, the stent is annealed at 120° C. for 74 hours and then rinsed with solvent.

The stent thus pretreated is placed for 45 minutes into a 1:1 mixture of 0.4 M EDC and 0.1 M NHS in Millipore water. Subsequently, it is briefly rinsed with Millipore water and dried in a stream of nitrogen.

Onto these stents, a solution of CD 133 (50 μg/ml) in buffer (free from amino acid) is placed and shaken overnight at 4° C.

The stents were rinsed with buffer in order to wash away non-bound protein.

The detection of bound protein took place by fluorescence labelled antigen CD 133.

What is claimed is:

1. A stent, comprising:
a) a basic stent body;
b) at least one anchor group on the surface of the basic stent body; and
c) at least one biomolecule which is bound to the at least one anchor group,
wherein the at least one anchor group is selected as the same or different from the group of compounds consisting of the general formula (I)
(R²O)₂(O)P-L-(CH₂)ₓ-M-R¹ (I)
wherein
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂—CH₂—O)_{y},
x represents an integer from 1 to 25 and
y represents an integer from 1 to 25.

2. The stent of claim 1, wherein the biomolecule or biomolecules are selected as the same or different from the group consisting of proteins, enzymes, growth factors, antibodies, and peptide sequences.

3. The stent of claim 1, wherein the at least one anchor group is selected as the same or different from the group of compounds consisting of the general formula (I) wherein
M represents —(CH₂—CH₂—O)_{y} and
y represents an integer from 1 to 25.

4. The stent of claim 1 wherein the basic stent body is selected from permanent or degradable metals or degradable polymer materials.

5. A method for producing a stent coated with biomolecules, the method comprising:
a) providing at least one basic stent body;
b) purifying the at least one basic stent body; and
c) selecting at least one identical or different anchor groups from the group of compounds consisting of the general formula (I)
(R²O)₂(O)P-L-(CH₂)ₓ-M-R¹ (I)
wherein
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂—CH₂—O)_{y},
x represents an integer from 1 to 25, and
y represents an integer from 1 to 25,
d) functionalizing the purified basic stent body from step b) with at least one identical or different anchor groups selected from the group of compounds consisting of the general formula (I) from step c);
e) providing at least one identical or different biomolecules selected from the group consisting of compounds which promote the attachment of the endothelial progenitor cells to the stent surface; and
f) binding at least one identical or different biomolecules from step e) to the functionalized at least one basic stent body from step d).

6. The method of claim 5, further comprising:
g) purifying the at least one stent of step b) in an oxygen plasma or rinsing the at least one stent of step b) with a solvent selected from the group consisting of dichloromethane, acetone, methanol and Millipore water; and
h) drying the at least one stent, as necessary.

7. The method of claim 5, further comprising:
i) providing at least one anchor groups selected from compounds consisting of the formula (I) according to step c) in at least one solvent selected from the group consisting of methanol, ethanol, acetone, tetrahydrofuran, dimethyl formamide, chloroform, dimethyl sulphoxide and dichloromethane;
j) bringing the purified at least one stent according to step b) into contact at least partially with the solution of the anchor group of the compounds with the formula (I);
k) evaporating at least a portion of the solvent as necessary;
l) thermally treating the at least one stent with at least one anchor group;
m) rinsing the at least one stent with at least one solvent, as necessary; and
n) drying the at least one stent, as necessary.

8. The method of claim 5, further comprising:
o) providing the biomolecule or biomolecules of step e) in at least one buffer which is free from amino acid;
p) bringing the functionalized at least one stent into contact at least partially with the same or different dissolved biomolecules;
q) rinsing the functionalized at least one stent, as necessary; and
r) drying the functionalized at least one stent, as necessary.

9. An anchor group for biomolecules on basic stent bodies, comprising:
a compound with the general formula (I)
(R²O)₂(O)P-L-(CH₂)ₓ-M-R¹ (I)
in which
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂, —CH₂—O)_{y},
x represents an integer from 1 to 25, and
y represents an integer from 1 to 25.

10. A method for producing at least one stent, comprising:
providing at least one stent,
incorporating at least one anchor group comprising a compound with the general formula (I)
(R²O)₂(O)P-L-(CH₂)ₓ-M-R¹ (I)
in which
R¹ represents —COOH, —OH, —SH, —NH₂, benzophenone or benzophenone derivatives,
R² represents hydrogen, —CH₂CH₃ or —CH₃,
L represents a single bond or —O—,
M represents a single bond or —(CH₂—CH₂—O)_{y},
x represents an integer from 1 to 25, and
y represents an integer from 1 to 25.
